(19) United States
(12) Patent Application Publication
(10) Pub. No.: US 2022/0028543 A1
(43) Pub. Date: Jan. 27, 2022

(54) DEVICE SAFETY BASED ON CONFLICTS IN PATIENT MEDICAL RECORDS

(71) Applicant: INTERNATIONAL BUSINESS MACHINES CORPORATION, Armonk, NY (US)

(72) Inventors: Robert Christian Sizemore, Fuquay-Varina, NC (US); David Blake Werts, Charlotte, NC (US); Sterling Richardson Smith, Apex, NC (US); Kristin E. McNeil, Charlotte, NC (US)

(21) Appl. No.: 16/935,409

(22) Filed: Jul. 22, 2020

(51) Int. Cl. G16H 40/67; G16H 10/60; G16H 50/30

(52) U.S. Cl. CPC G16H 40/67; G16H 50/30; G16H 10/60

(57) ABSTRACT

Aspects of the present disclosure include receiving, by a processor, a procedure code for a patient, wherein the procedure code is associated with a medical device, obtaining, by the processor, patient medical data for the patient, analyzing the patient medical data to identify one or more patient medical conditions and one or more other procedure codes, determining a risk score for the procedure code based on the one or more patient medical conditions and the one or more other procedure codes, and enacting an action based at least in part on the risk score for the procedure code

### BACKGROUND

The present invention generally relates to natural language processing, and more specifically, to device safety based on conflicts in patient medical records.

In the medical field, healthcare professionals will often see multiple patients a day. These professionals, during their interactions with patients, will establish a medical record for the patient based on observations, insights, treatments, diagnosis, and the like. This medical record is often in an unstructured format including natural language narrative summaries of the interaction with the patient. In addition, the medical record will include potential orders for testing and procedures to be performed by medical devices such as, for example, a magnetic resonance imaging (MM) device. When ordering this type of testing, healthcare professionals may not have reviewed the entirety of the medical record and patient's history. This can be due to the sheer volume of medical history data contained within the medical record.

Natural language processing (NLP) is a field of computer science, artificial intelligence, and linguistics concerned with the interactions between computers and human (natural) languages. As such, NLP is related to the area of human-computer interaction, and especially with regard to natural language understanding that enables computers to derive meaning from human or natural language input.

### SUMMARY

Embodiments of the present invention are directed to a computer-implemented method for device safety based on conflicts in the patient medical history. A non-limiting example of the computer-implemented method includes receiving, by a processor, a procedure code for a patient, wherein the procedure code is associated with a medical device, obtaining, by the processor, patient medical data for the patient, analyzing the patient medical data to identify one or more patient medical conditions and one or more other procedure codes, determining a risk score for the procedure code based on the one or more patient medical conditions and the one or more other procedure codes, and enacting an action based at least in part on the risk score for the procedure code.

Embodiments of the present invention are directed to a system for device safety based on conflicts in the patient medical history. A non-limiting example of the system includes a processor coupled to a memory, the processor configured to perform receiving, by the processor, a procedure code for a patient, wherein the procedure code is associated with a medical device, obtaining, by the processor, patient medical data for the patient, analyzing the patient medical data to identify one or more patient medical conditions and one or more other procedure codes, determining a risk score for the procedure code based on the one or more patient medical conditions and the one or more other procedure codes, and enacting an action based at least in part on the risk score for the procedure code.

Embodiments of the invention are directed to a computer program product for device safety based on conflicts in the patient medical history, the computer program product comprising a computer readable storage medium having program instructions embodied therewith. The program instructions are executable by a processor to cause the processor to perform a method. A non-limiting example of the method includes receiving, by a processor, a procedure code for a patient, wherein the procedure code is associated with a medical device, obtaining, by the processor, patient medical data for the patient, analyzing the patient medical data to identify one or more patient medical conditions and one or more other procedure codes, determining a risk score for the procedure code based on the one or more patient medical conditions and the one or more other procedure codes, and enacting an action based at least in part on the risk score for the procedure code.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment according to one or more embodiments of the present invention;

FIG. 2 depicts abstraction model layers according to one or more embodiments of the present invention;

FIG. 3 depicts a block diagram of a computer system for use in implementing one or more embodiments of the present invention;

FIG. 4 depicts a block diagram of a system for device safety based on risk assessments according to embodiments of the invention; and

FIG. 5 depicts a flow diagram of a method for device safety according to one or more embodiments of the invention.

The diagrams depicted herein are illustrative. There can be many variations to the diagram, or the operations described therein without departing from the spirit of the invention. For instance, the actions can be performed in a differing order or actions can be added, deleted or modified. Also, the term “coupled”, and variations thereof describes having a communications path between two elements and does not imply a direct connection between the elements with no intervening elements/connections between them. All of these variations are considered a part of the specification.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

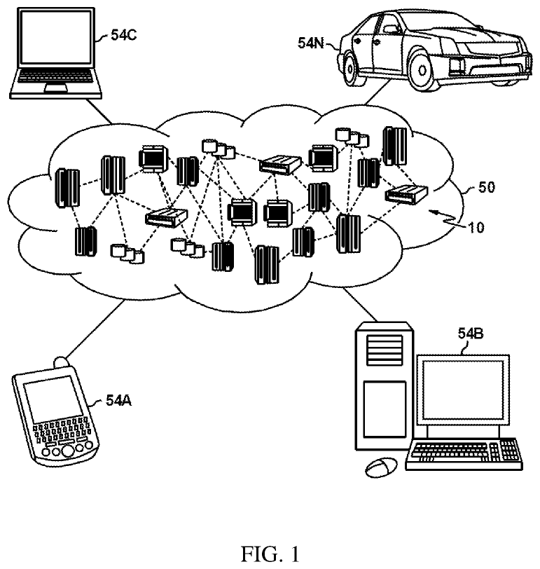

Referring now to FIG. 1, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

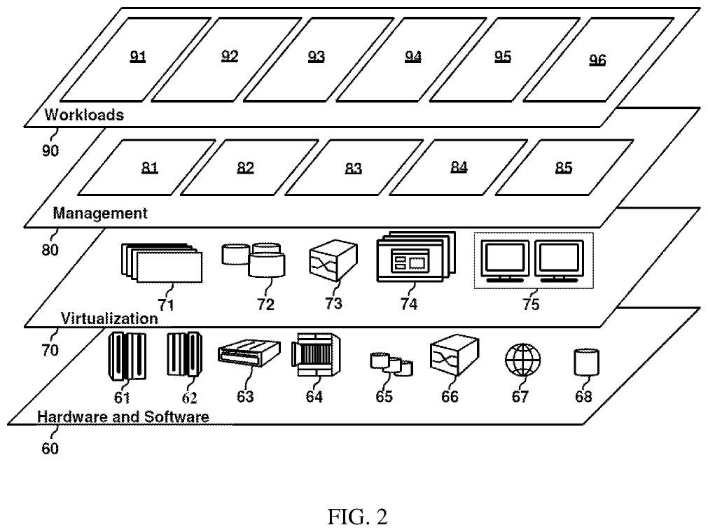

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and medical device safety based on conflicts in a patient medical record 96.

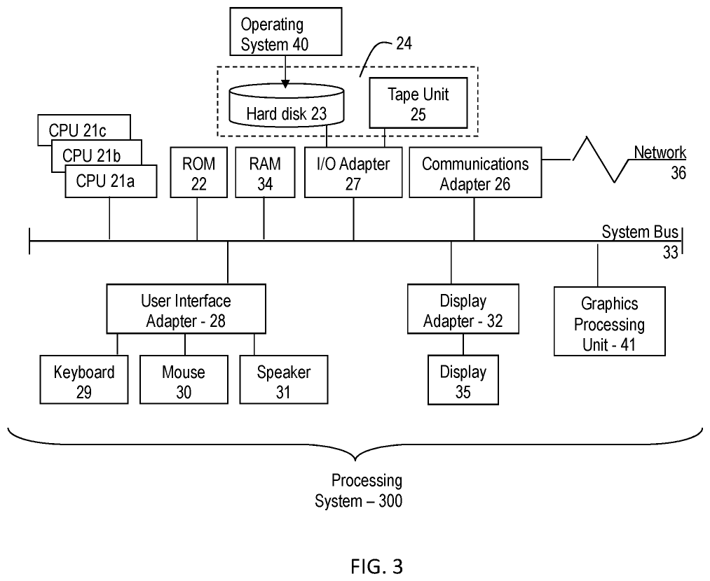

Referring to FIG. 3, there is shown an embodiment of a processing system 300 for implementing the teachings herein. In this embodiment, the system 300 has one or more central processing units (processors) 21a, 21b, 21c, etc. (collectively or generically referred to as processor(s) 21). In one or more embodiments, each processor 21 may include a reduced instruction set computer (RISC) microprocessor. Processors 21 are coupled to system memory 34 and various other components via a system bus 33. Read only memory (ROM) 22 is coupled to the system bus 33 and may include a basic input/output system (BIOS), which controls certain basic functions of system 300.

FIG. 3 further depicts an input/output (I/O) adapter 27 and a network adapter 26 coupled to the system bus 33. I/O adapter 27 may be a small computer system interface (SCSI) adapter that communicates with a hard disk 23 and/or tape storage drive 25 or any other similar component. I/O adapter 27, hard disk 23, and tape storage device 25 are collectively referred to herein as mass storage 24. Operating system 40 for execution on the processing system 300 may be stored in mass storage 24. A network adapter 26 interconnects bus 33 with an outside network 36 enabling data processing system 300 to communicate with other such systems. A screen (e.g., a display monitor) 35 is connected to system bus 33 by display adaptor 32, which may include a graphics adapter to improve the performance of graphics intensive applications and a video controller. In one embodiment, adapters 27, 26, and 32 may be connected to one or more I/O busses that are connected to system bus 33 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 33 via user interface adapter 28 and display adapter 32. A keyboard 29, mouse 30, and speaker 31 all interconnected to bus 33 via user interface adapter 28, which may include, for example, a Super I/O chip integrating multiple device adapters into a single integrated circuit.

In exemplary embodiments, the processing system 300 includes a graphics processing unit 41. Graphics processing unit 41 is a specialized electronic circuit designed to manipulate and alter memory to accelerate the creation of images in a frame buffer intended for output to a display. In general, graphics processing unit 41 is very efficient at manipulating computer graphics and image processing and has a highly parallel structure that makes it more effective than general-purpose CPUs for algorithms where processing of large blocks of data is done in parallel.

Thus, as configured in FIG. 3, the system 300 includes processing capability in the form of processors 21, storage capability including system memory 34 and mass storage 24, input means such as keyboard 29 and mouse 30, and output capability including speaker 31 and display 35. In one embodiment, a portion of system memory 34 and mass storage 24 collectively store an operating system coordinate the functions of the various components shown in FIG. 3.

Turning now to an overview of technologies that are more specifically relevant to aspects of the invention, in the medical field, doctors and other healthcare professionals may interact with several patients each day. During these interactions, the doctors need to make fast and accurate decisions on the types of treatment or types of tests that are needed to be performed. When making determinations about the types of tests and treatments, a doctor needs to mindful of several criteria including medical test costs, patient symptoms, patient diagnosis and disease states, and prior procedures, treatments, and medical tests. These criteria need to be considered when determining whether there may be a medical conflict for the patient and the proposed procedure and/or test. However, a doctor may not have access to all this information in a patient's medical history when prescribing a test or treatment by a medical device.

Turning now to an overview of the aspects of the invention, one or more embodiments of the invention address the above-described shortcomings of the prior art by providing a system to determine medical device safety based on natural language processing (NLP) on medical unstructured text (e.g., patient medical records). The NLP on the medical unstructured text identifies a patient's medical conditions and assists with determining a risk score for the patient for performing a test using a medical device. The test can be associated with a procedure code ordered by a doctor or other healthcare professional. The procedure code can be in any format and is deterministic of the type of test or procedure that will be performed by a medical device. If the system determines that a safety risk is present, the system can perform an action including, but not limited to, generating a warning, stopping the performance of the medical device requiring an override, adjusting parameters of the medical device, or generating an alert. The NLP on the unstructured medical texts produces insights into the text that can be utilized for determining this risk score for the patient. For example, if a patient has a pacemaker which is indicated in the patient's medical history, then the patient would be at risk if an MRI test procedure code was ordered and the MRI performed. As the MRI is being configured for the patient, the MRI device can initiate NLP on the medical record to determine the risk score for the patient. The MRI device can compare the risk score to various defined threshold to determine what action to take.

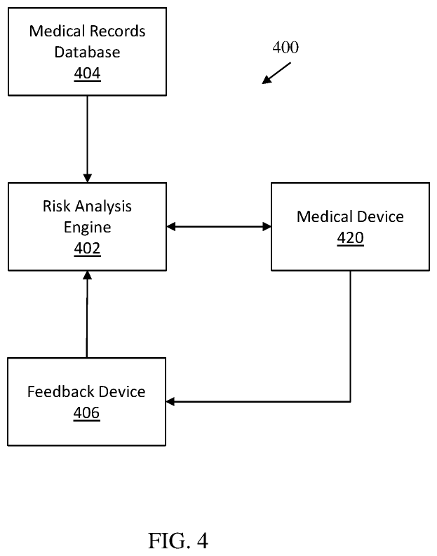

Turning now to a more detailed description of aspects of the present invention, FIG. 4 depicts block diagram of a system for device safety based on risk assessments according to embodiments of the invention. The system 400 includes a risk analysis engine 402 and a medical device 420. The medical device 420 can be any type of device that can perform medical measurements and/or tests for a patient. As mentioned above, the risk analysis engine 402 can utilize a variety of techniques to analyze a medical records database 404. Medical records database 404 can include patient's medical records including diagnosis, treatment, prescription information, test results, medical history, and patient demographic and biographical information. Typically, this data is in an unstructured text in the form of physician, pharmacist, and lab technician notes and the like. The risk analysis engine 402 performs text analytics on the medical records using machine learning and/or a rules/dictionary based NLP to gather insights into the medical record such as, for example, patient medical conditions. In one or more embodiments of the invention, the risk analysis engine 402 can utilize a dictionary to first analyze the text and define and label entities within the text. These entities are words and phrases in the text that can be labelled using any suitable labelling rules including, but are not limited to, diagnosis, medical condition, treatments, medications, prescriptions, disease, disease state, and disease status. These entities can overlap, and an entity can have multiple labels or annotations applied to them. For example, a diagnosis of “uncontrolled high blood pressure” can be labelled as a diagnosis, medical condition, disease, disease state, and disease status.

In other embodiments of the invention, the risk analysis engine 402 can utilize machine learning techniques to label and annotate entities in the medical records using any suitable vector formation and clustering technique to represent each training/validation set entity (word or phrase) in vector form and then determine a similarity or grouping of different vectors, such as by using a neural network language model representation techniques (e.g., Word2Vec, Doc2Vec, or similar tool) to convert words and phrases to vectors which are then input to a clustering algorithm to place words and phrases with similar meanings close to each other in a Euclidean space.

In one or more embodiments of the invention, the risk analysis engine 402 can annotate the medical records from the medical records database 404 and store this annotated record in a memory associated with the risk analysis engine 402, a memory associated with the medical device 420, or in the database 404.

In one or more embodiments of the invention, the medical device 420 can be any device utilized for taking measurements or performing tests on a patient. Often, these devices, utilize techniques that can be invasive to the patient such as, for example, an X-ray device which emits high energy electromagnetic radiation at a patient for the purpose of projectional radiography. Certain characteristics of a patient can make the usage of an X-ray device dangerous to a specific patient due to medical conflicts that the system 400 can find in the patient's medical records. When a patient is prescribed a test by a medical device 420, the annotated patient data can be utilized as an input to the medical device 420. The risk analysis engine 402 (which can be located on a processing device within the medical device 420 or can be in a separate processing device, see FIG. 3) analyzes the patient medical data that has been annotated and compares this to characteristics of the medical device 420. A conflict list can be developed by the risk analysis engine 402 for any medical conflicts between the patient and the medical device 420. For each medical conflict in the conflict list, an associated risk score can be determined based on the medical risk for the patient in using the medical device 420. This risk score can be compared to a pre-defined threshold to determine the associated risk to the patient. In one or more embodiments of the invention, based on the risk score exceeding a threshold value, the medical device 420 and/or the risk analysis engine 402 can enact an action on the medical device 420. These actions include, but are not limited to, generating, transmitting, and/or displaying an alert to a technician, doctor, and/or patient, shutting down the medical device 420, adjusting a parameter for the medical device 420, and requesting updated medical information for the patient. When transmitting an alert to a doctor or technician, the alert can include an option for the doctor or technician to override the alert and authorize the test or procedure for the medical device 420. In one or more embodiments of the invention, the risk score can be compared to multiple thresholds that each have a different action that can be enacted by the medical device 420 and/or the risk analysis engine 402. For example, if a risk score is above a first threshold but below a second threshold, the action can be to request additional authorization from the doctor or technician that ordered the test or procedure. If the risk score exceeds the first and second threshold, for example, the action can be to shut down the medical device 420 so that it may not perform the test or procedure. If the risk score is below the predefined threshold then the medical device 420 can operate normally.

In one or more embodiments of the invention, the analysis of the patient medical records can identify previous procedure codes that show that a patient has undergone one or more other procedures associated with various medical devices. For example, if a patient medical record shows that a patient has had multiple X-ray procedures within a year and the patient has been scheduled to have another X-ray. The risk analysis engine 402 would indicate this with the risk score for the patient and an action could be taken. In one or more embodiments of the invention, the action enacted for the medical device 420 can be the modification of the device behavior. In the above example, based on the risk score compared to various pre-defined thresholds, the action taken for the device would be to lower the radiation levels of the x-ray emissions or any other adjustment to parameters on the device to mitigate the risk to the patient based on the risk score derived from the patient medical records.

In one or more embodiments of the invention, the system 400 includes a feedback device 406 that can deliver additional environmental data and physiological data associated with the test location, the patient, and/or both. The physiological data can be taken by the feedback device 406 in the form of a physiological sensor for the patient. For example, if a patient is receiving dialysis treatment from the medical device 420, the feedback device 406 can continuously monitor physiological information about the patient such as, for example, blood pressure, temperature, pulse rate, oxygen levels, and the like. This physiological data taken in real-time or close to real-time can be analyzed by the risk analysis engine 402 during the testing and the risk score can be adjusted. As the risk score is adjusted, the action enacted by the risk analysis engine 402 can change. In the dialysis example, if the patient's blood pressure exceeds a certain value, then the risk analysis engine 402 can shut down the medical device 420 performing the dialysis. In addition to the physiological data, the feedback device 406 can provide environmental data associated with the test or procedure performed by the medical device 420. Environmental data can include weather data, air pressure, humidity, various gas detection data, water purity sensor data, and the like. The environmental data can be analyzed by the risk analysis engine 402 to adjust the risk score for the test or procedure. In the dialysis example above, the feedback device 406 can be a water sensor that detects water purity. The sensor can indicate impurities in the water utilized for the dialysis procedure and the risk analysis engine 402 can utilize this sensor data to determine a new action to enact or adjust the current action. An example action could be the generating an alert to a water purifying technician or shutting down the medical device 420.

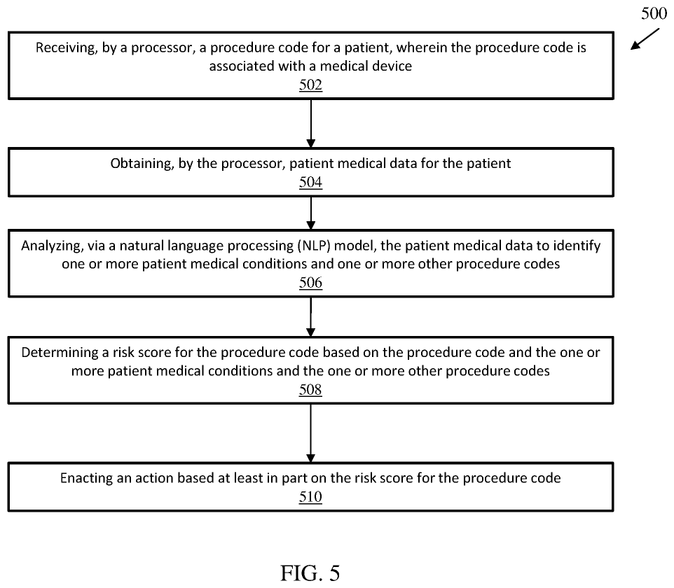

FIG. 5 depicts a flow diagram of a method for device safety according to one or more embodiments of the invention. The method 500 includes receiving, by a processor, a procedure code for a patient, wherein the procedure code is associated with a medical device, as shown in block 502. The procedure code can originate through an order for a test or procedure from a doctor or other healthcare professional. At block 504, the method 500 includes obtaining, by the processor, patient medical data for the patient. Also, at block 506, the method 500 includes analyzing the patient medical data to identify one or more patient medical conditions and one or more other procedure codes. The method 500, at block 508, also includes determining a risk score for the procedure code based on the procedure code and the one or more patient medical conditions and the one or more other procedure codes. And at block 510, the method 500 includes enacting an action based at least in part on the risk score for the procedure code.

Additional processes may also be included. It should be understood that the processes depicted in FIG. 5 represent illustrations, and that other processes may be added, or existing processes may be removed, modified, or rearranged without departing from the scope and spirit of the present disclosure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving, by a processor, a procedure code for a patient, wherein the procedure code is associated with a medical device;
obtaining, by the processor, patient medical data for the patient;
analyzing the patient medical data to identify one or more patient medical conditions and one or more other procedure codes;
determining a risk score for the procedure code based on the one or more patient medical conditions and the one or more other procedure codes; and
enacting an action based at least in part on the risk score for the procedure code.

2. The computer-implemented method of claim 1, further comprising:
receiving updated patient medical data;
analyzing the updated patient medical data to determine a change in the one or more patient medical conditions; and
updating the risk score based on the change in the one or more patient medical conditions.

3. The computer-implemented method of claim 2, wherein the updated patient medical data comprises data associated with a procedure performed on the patient by the medical device.

4. The computer-implemented method of claim 2, wherein the updated patient medical data comprises physiological data, from a physiological sensor, for the patient.

5. The computer-implemented method of claim 1, wherein the action comprises disabling a medical device associated with the procedure code.

6. The computer-implemented method of claim 1, wherein the action comprises generating and transmitting an alert.

7. The computer-implemented method of claim 1, wherein the action comprises requesting updated medical data for the patient.

8. A system comprising:
a processor communicatively coupled to a memory, the processor configured to:
receive a procedure code for a patient, wherein the procedure code is associated with a medical device;
obtain patient medical data for the patient;
analyze the patient medical data to identify one or more patient medical conditions and one or more other procedure codes;
determine a risk score for the procedure code based on the one or more patient medical conditions and the one or more other procedure codes; and
enact an action based at least in part on the risk score for the procedure code.

9. The system of claim 8, wherein the processor is further configured to:
receive updated patient medical data;
analyze the updated patient medical data to determine a change in the one or more patient medical conditions; and
update the risk score based on the change in the one or more patient medical conditions.

10. The system of claim 9, wherein the updated patient medical data comprises data associated with a procedure performed on the patient by the medical device.

11. The system of claim 9, wherein the updated patient medical data comprises physiological data, from a physiological sensor, for the patient.

12. The system of claim 8, wherein the action comprises disabling a medical device associated with the procedure code.

13. The system of claim 8, wherein the action comprises generating and transmitting an alert.

14. The system of claim 8, wherein the action comprises requesting updated medical data for the patient.

15. A computer program product for device safety comprising a computer readable storage medium having program instructions embodied therewith, the program instructions executable by a processor to cause the processor to perform a method comprising:
receiving, by the processor, a procedure code for a patient, wherein the procedure code is associated with a medical device;
obtaining, by the processor, patient medical data for the patient;
analyzing the patient medical data to identify one or more patient medical conditions and one or more other procedure codes;
determining a risk score for the procedure code based on the one or more patient medical conditions and the one or more other procedure codes; and
enacting an action based at least in part on the risk score for the procedure code.

16. The computer program product of claim 15, further comprising:
receiving updated patient medical data;
analyzing the updated patient medical data to determine a change in the one or more patient medical conditions; and
updating the risk score based on the change in the one or more patient medical conditions.

17. The computer program product of claim 16, wherein the updated patient medical data comprises data associated with a procedure performed on the patient by the medical device.

18. The computer program product of claim 16, wherein the updated patient medical data comprises physiological data, from a physiological sensor, for the patient.

19. The computer program product of claim 15, wherein the action comprises disabling a medical device associated with the procedure code.

20. The computer program product of claim 15, wherein the action comprises generating and transmitting an alert.
